(19) United States
(12) Patent Application Publication
(10) Pub. No.: US 2009/0227816 A1
(43) Pub. Date: Sep. 10, 2009

(54) Process for preparing polybrominated compounds

(75) Inventors: Haim Stollar, Omer (IL); Gershon Miaskovski, Be'er Sheva (IL); Aharon Meirom, Arad (IL); Michael Peled, Be'er Sheva (IL); Jie Yu, Shanghai (CN); Iris Ben David, Ashdod (IL)

Correspondence Address: NIXON & VANDERHYE, PC, 901 NORTH GLEBE ROAD, 11TH FLOOR, ARLINGTON, VA, 22203, US

(73) Assignee: BROMINE COMPOUNDS LTD., Be'er-Sheva (IL)

(21) Appl. No.: 12/379,707

(22) Filed: Feb. 26, 2009

Related U.S. Application Data
(63) Continuation-in-part of application No. PCT/IL2007/001077, filed on Aug. 30, 2007.
(60) Provisional application No. 60/841,207, filed on Aug. 31, 2006.

(30) Foreign Application Priority Data
Jun. 10, 2007 (IL) 183811

(51) Int. Cl. C07C 41/01; C07C 17/013; C07C 25/18

(52) U.S. Cl. 568/639; 570/206; 570/184

(57) ABSTRACT

A process for preparing a polybrominated product such as decabromodiphenyl ether and decabromodiphenyl ethane, which comprises brominating a reduced particle size precursor of said polybrominated product in an organic solvent or in bromine as a solvent, wherein the bromination is carried out either concurrently with or subsequent to said particle size reduction, forming the polybrominated product and separating the same from the reaction mixture.

Polybrominated aromatic compounds such as decabromodiphenyl ether and decabromodiphenyl ethane are used as flame retardants for, inter alia, plastics and textile applications. These compounds are produced by brominating diphenyl ether (DPO; also named herein diphenyl oxide) and diphenyl ethane (DPE), respectively, in the presence of a Lewis acid catalyst, most commonly aluminum chloride, in a suitable solvent, which is preferably a halogenated hydrocarbon such as dichloromethane (DCM), bromochloromethane (CBM) and dibromomethane (DBM), and most preferably, in a mixture thereof. Alternatively, the bromination reaction is carried out using bromine as the solvent.

During the bromination process, the desired polybrominated product precipitates from the reaction solution. The higher the degree of bromination, the lower the solubility of the polybrominated product. Obviously, there will always be present a certain amount of underbrominated species, of higher solubility, that will be entrapped in the crystal of the polybrominated product (possibly together with solvent molecules). Thus, the desired polybrominated product, namely, decabromodiphenyl ether or decabromodiphenyl ethane, may contain significant quantities of underbrominated species such as nonabromodiphenyl ether (Nona) and nonabromodiphenyl ethane, respectively, and even lower brominated derivatives.

The purity profile of the currently commercially available decabromodiphenyl ether is typically as follows: the product comprises about 97.5 to 98.5% decabromodiphenyl ether, with the remaining 1.5-2.5% consisting mostly of two nonabromodiphenyl ether (Nona) isomers. Unfortunately, on an industrial scale, neither prolonged reaction periods using a large excess of the brominating reagents, nor common purification methods, and specifically, re-crystallization procedures, are capable of improving the purity profile of decabromodiphenyl ether. Specifically, in the case of decabromodiphenyl ether, re-crystallization procedures were found to be ineffective, since the solubility of decabromodiphenyl ether and the underbrominated derivatives contaminating the same (namely, nonabromodiphenyl ether) are very low in most solvents, and, furthermore, these solubilities are very similar to one another. Furthermore, the purification of decabromodiphenyl ethane by means re-crystallization is even more difficult, because of its especially low solubility.

U.S. Pat. No. 3,752,856 describes a process for brominating aromatic compounds in the absence of a solvent by crushing the crystalline reaction mass. The publication reports that diphenyl oxide is reacted with a slight stoichiometric excess of bromine in the absence of a solvent, and following recrystallization in trichlorobenzene, the product obtained has a melting point of 294-295° C.

It has now been found that it is possible to obtain a polybrominated product having improved purity profile and, more specifically, highly pure polybrominated product which is substantially free from the corresponding underbrominated derivatives (e.g., decabromodiphenyl ether product containing less then 1.0% by weight nonabromodiphenyl ether) by reducing the particle size of a precursor of the polybrominated product, either prior to, or essentially concurrently with, the reaction of said precursor with the brominating agent in an organic solvent or in bromine as a solvent. It has been found that despite the fact that crystals of the polybrominated product (decabromodiphenyl ether), tend to rapidly grow when obtained following the bromination reaction in a solvent or in bromine as a solvent, especially under heating, it is still possible, by means of using a pre-milled precursor with an appropriate particle size distribution and/or employing suitable in-situ milling conditions, to accomplish the bromination reaction sufficiently rapidly, thus improving the level of purity of the polybrominated product.

In the context of the present invention, the term “polybrominated product” refers to an aromatic compound where the free aromatic sites are fully brominated, as illustrated by the structure depicted below for two particularly preferred compounds according to the present invention:

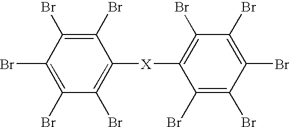

wherein X is —O— or —CH₂—CH₂— (for decabromodiphenyl ether and decabromodiphenyl ethane, respectively), which product contains less than 15% of the corresponding underbronminated derivatives, and more preferably less than 10% of said derivatives (usually by area % by HPLC or gas chromatography; throughout the description, the purity of the polybrominated product is expressed in terms of area % in respect to the predominant, fully brominated compound, and in terms of weight % in respect to the underbrominated impurity (nonabromodiphenyl ether), when the content of said impurity is not more than about 1%).

The term “highly pure polybrominated product” and the like, as used herein, relates to a polybrominated product, as described above, which is substantially free from underbrominated derivatives, which product comprises not less then 99.0%, and preferably not less then 99.3%, and more preferably not less then 99.4%, of the fully brominated aromatic compound. More specifically, with reference to decabromodiphenyl ether, the term “highly pure product” refers to a product comprising less than 1.0%, and preferably less than 0.8%, and most preferably less than 0.7% nonabromodiphenyl ether.

Analytical methods which may be used for determining the purity profile of the polybrominated products prepared by the process of the present invention include qualitative gas chromatography (applicable for decabromodiphenyl ethane) and qualitative and quantitative HPLC (applicable for decabromodiphenyl ether). The analytical methods are described in more detail below.

As used herein, the term “precursor of a polybrominated product” refers to a compound, or a mixture of compounds, which are transformable by means of a bromination reaction to a polybrominated product having the desired degree of purity, and which are insoluble in the liquid phase of the reaction mixture under the conditions of the bromination reaction. Thus, the term “precursor of a polybrominated product” specifically includes one or more underbrominated derivatives which are insoluble under the conditions of the bromination reaction (e.g., hepta, octa and nona-bromo diphenyl ether or hepta, octa and nona-bromo diphenyl ethane, which derivatives precipitate from the liquid phase upon brominating the corresponding non-brominated starting materials (diphenyl ether or diphenyl ethane, respectively)), or any brominated composition which comprises the fully brominated compound together with unacceptable amounts of the aforementioned undesired underbrominated derivatives thereof (namely, a composition comprising more than 1.1% of the underbrominated derivatives).

By the term “reduced particle size precursor” is meant a precursor, as defined hereinabove, which was subjected to particle size reduction (e.g., by milling, as described in more detail below).

We found that decabromodiphenyl ether with a purity assay of about 97.5% to about 98.5%, which is the material normally resulting from the synthesis according to the prior art processes, can be used according to the present invention as a precursor for obtaining high purity decabromodiphenyl ether product. More specifically, if the aforementioned decabromodiphenyl ether precursor is milled to produce particles having particle size below about 14 μm (d₉₀), and is subsequently brominated, then the resulting decabromodiphenyl ether product has an assay greater than 99% with a nonabromodiphenyl ether content less than 1%.

Alternatively, we found that decabromodiphenyl ether product which is substantially free from nonabromodiphenyl ether, or decabromodiphenyl ethane with reduced underbrominated impurities such as nonabromodiphenyl ethane impurities, respectively, can also be produced in a one step process, whereby the corresponding diphenyl oxide or diphenyl ethane starting materials are brominated in a suitable solvent or in bromine, and insoluble underbrominated derivatives which precipitate from the liquid phase are milled while the bromination reaction is still in progress. In this way a precursor having a reduced particle size suitable for enhanced bromination is in-situ formed within the reaction mixture and is immediately transformed into the final polybrominated product.

Accordingly, in a first aspect, the present invention provides a process, which comprises brominating a reduced particle size precursor of a polybrominated product in an organic solvent or in bromine as a solvent, wherein the bromination is carried out either concurrently with or subsequent to said particle size reduction, forming the polybrominated product and separating the same from the reaction mixture.

It should be noted that the reduced particle size precursor to be brominated according to the present invention may be a pre-milled precursor composed of sufficiently small particles, as will be described quantitatively in more detail below, in which case it is not necessary to reduce the particle size of the precursor's particles in the reaction mixture during the bromination reaction. According to this variant of the invention, the pre-milled precursor consists mostly of the polybrominated compound (e.g., with a purity assay of about 95% to 98.5%, and more specifically, 97% to 98.5% decabromodiphenyl ether, and typically not less than 1.1% nonabromodiphenyl ether), and is capable of being transformed, following the bromination, to the highly pure product in which the content of the underbrominated derivatives is less than 1.0%.

In another embodiment of the invention, an aromatic starting material in a liquid form may be used, which is converted into a solid brominated precursor during the reaction, in which case the particle size of the precursor's particles may be reduced essentially simultaneously with the bromination reaction.

Alternatively, a suitable precursor may be isolated from the reaction mixture in a solid form before the bromination reaches completion, subjected to particle size reduction, and then brominated to arrive at the desired polybrominated product.

According to one preferred embodiment, the polybrominated product is a highly pure decabromodiphenyl ether and the precursor used for obtaining the same is decabromodiphenyl ether material contaminated with more than 1.1-1.5% of the nonabromodiphenyl ether impurity. Alternatively, the precursor comprises one or more underbrominated derivatives of diphenyl oxide or a mixture thereof with decabromodiphenyl ether, which are preferably formed in-situ upon brominating diphenyl oxide.

According to another preferred embodiment, the polybrominated product is decabromodiphenyl ethane product which contains not less than 85%, and preferably not less than 90% of the decabromodiphenyl ethane compound, and more preferably between: 95% and 99% of the decabromodiphenyl ethane compound. The precursor used for obtaining the aforementioned decabromodiphenyl ethane product comprises one or more underbrominated derivatives of diphenyl ethane or a mixture thereof with decabromodiphenyl ethane, which are preferably formed in-situ upon brominating diphenyl ethane.

The reduction of the particle size of the precursor's particles may be accomplished using numerous known methods, and especially, by means of various forms of milling, including, for example, ball milling, fluid energy milling, roller milling and ultrasound sonicators.

According to one embodiment, the milling is carried out in the reaction vessel concurrently with the bromination reaction using appropriate mechanical means, for example, suitable impellers and baffles to increase turbulence, and/or in the presence of rigid grinding media, namely, abrasive materials such as ceramic or glass beads. Alternatively, ultrasound energy is applied in order to produce small particles of the precursor.

Alternatively, the milling of the precursor's particles is carried out outside the reaction vessel. For example, the reaction mixture may be passed through a milling device or an ultrasonic device placed outside the reaction vessel, with the milled material returned to the reaction vessel.

Alternatively, the precursor may be isolated from the liquid phase at any stage during the bromination reaction, subjected to milling using the methods described above, and returned to the reaction vessel in order to complete the bromination reaction and to afford the desired polybrominated product having the contemplated purity profile.

The size distribution of the particles may be determined by known techniques, such as, for example, light scattering, laser diffraction or microscopy. The particle size distribution may be described using either the d₁₀, d₅₀ and d₉₀ parameters (a particle size distribution of dₓ is defined as a distribution where x percent by volume of the particles are smaller than the size indicated).

According to another embodiment of the invention, the particle size distribution of a reduced particle size precursor that may be brominated according to the present invention (that is, a pre-milled precursor) is characterized by d₉₀ not greater than 14 microns and preferably not greater than 4 microns. For example, a suitable precursor is decabromodiphenyl ether material which comprises about 97.0 to 98.5% decabromodiphenyl ether, whose particles population is characterized by d₉₀ value not greater than 14 μm (this precursor is obtained upon milling, e.g., by means of air-jet milling, decabromodiphenyl ether resulting from known synthetic methods). As illustrated in the examples below, this reduced particle size precursor may be brominated to give highly pure decabromodiphenyl ether which is substantially free from the nanobromodiphenyl ether impurity.

Regarding the bromination reaction that is performed in halogenated hydrocarbons starting from diphenyl ether or diphenyl ethane, it is typically conducted by adding DPO at a temperature below 10° C. or by adding DPE at about 20-30° C. to the mixture of solvents, as described above, bromine and catalyst followed by heating at reflux to complete the reaction. More specifically, when the starting material is DPO, the reaction is carried out at temperature below 10° C. and post reaction heating at about the reflux temperature. For DPE, the reaction may be carried out at 20-30° C. and higher. The concentration of the precursor is preferably 0.5 to 1.1 kg per liter of solvent mixture. Suitable catalysts that may be used are Lewis acids such as AlCl₃, AlBr₃, SbCl₃, SbBr₃, Sb₂O₃, FeCl₃, FeBr₃, ZnCl₂ and BF₃, with AlCl₃ being particularly preferred. The catalyst is normally used at a concentration of about 10 to 27 g of catalyst per 100 g of DPO or DPE. Temperature values indicated throughout this specification apply for a reaction performed under atmospheric pressure; such temperature values may therefore vary according to pressure variation.

The bromination reaction that is performed in halogenated hydrocarbons starting from a pre-milled decabromodiphenyl ether can be conveniently conducted by preparing a mixture of the pre-milled material, catalyst and bromine in said halogenated hydrocarbons at 5-30° C. and heating the mixture at the reflux temperature.

Regarding the bromination reaction that is performed in bromine as a solvent starting from diphenyl ether or diphenyl ethane, it is typically conducted similarly to the procedures given above. In the context of the present invention, the term “bromine as a solvent” indicates that bromine is present in a sufficient amount relative to the solid precursor so as to form a stirrable mixture. Preferably, 0.5 to 0.9 kg precursor per liter of bromine are used, such that bromine is present in a molar excess of not less than about 80%. More specifically, in the case of making decabromodiphenyl ethane, the molar ratio between the total amount of bromine used diphenylethane may vary in the range between 18:1 and 30:1.

In the case wherein the precursor is concurrently milled and brominated, then the polybrominated product resulting from the bromination reaction consists of particles having small particle size (for example, a characteristic particle size distribution has d₉₀ value not greater than 14 microns). This decabromodiphenyl ether product is not easily separable from the reaction mixture. We found that the isolation of the decabromodiphenyl ether from the reaction mixture may be considerably facilitated upon heating the same in a solvent, preferably in one or more halogenated hydrocarbons as those mentioned hereinabove, thereby enlarging the particles of the product and improving its filterability. Accordingly, the isolation of the decabromodiphenyl ether may be effectively accomplished by stopping the milling operation, heating the reaction mixture, preferably to a temperature in the range of 35 to 70° C. for about 2 to 6 hours and finally isolating the same from the liquid phase, preferably by filtration.

If desired, the aforementioned product may be milled to give a polybrominated product which satisfies the desired purity profile (e.g., decabromodiphenyl ether containing less than 1% nanobromodiphenyl ether, or decabromodiphenyl ethane product containing less than 10% impurities) and which is in the form of sufficiently small particles suitable for use as flame retardant.

A method which was found to be especially suitable for large scale production comprises the bromination of diphenyl ether under mixing by means of one or more high axial flow impellers, whereby the particle size of the in-situ formed solid precursor is reduced and highly pure decabromodiphenyl ether may be recovered. High axial flow impellers generate vertical flow and radial flow within the reactor. Axial flow impellers are described in U.S. Pat. No. 4,468,130, U.S. Pat. No. 4,722,608, U.S. Pat. No. 4,896,971 and EP 469302. An especially suitable impeller that meets the aforementioned requirement is described in EP 1038572, which is incorporated herein by reference. Briefly, the impeller used in the bromination reaction comprises a hub having a bore extending therethrough for receiving a drive shaft therein, and two or more variable pitch blades that outwardly project from said hub, essentially in a radial direction. Each blade has a first edge that is contiguous with the hub (hereinafter the “near edge”), an opposite distant edge, and a leading edge and a trailing edge connecting said near and distant edges (these terms are used to indicate the first and last edges of the blade that contact the fluid upon rotating the impeller, respectively), with the blades being smoothly tapered in shape, wherein the width of the blade increases from the near edge to the distant edge, such that the ratio between the length of the near edge and the distant edge is in the range between 1:1.5 to 1:2.5. The angle of inclination of the blade with respect to the central axis of the hub (the axis of rotation) is position dependent. At the near edge, the angle of inclination (designated a) is smaller than the angle of inclination measured at the distant edge (designated β). Typically, α is in the range between 45° and 60° whereas β is in the range between 50° and 70°, with the difference therebetween being in the range of 6 to 12°. Such impellers are commercially available (MaxFlo turbine, manufactured by Pfaudler Company).

A preferred agitator assembly may include one impeller mounted on a shaft, said impeller having four glass-coated blades that preferably symmetrically project (90° apart) from the hub. Alternatively, multi-hubbed separable blade agitators may be used, wherein, for example, two impellers, each comprising a pair of blades as described above are mounted on a shaft with a small or minimal vertical separation between the hubs of said two impellers, thus providing an arrangement of four glass-coated blades with an adjustable angular separation there between. More specifically, for a large capacity reactor (about 10 to 20 cubic meter volume) it is preferred to use an agitator assembly according to each of the embodiments described above, with an additional impeller (e.g., a curved blade turbine) mounted below the axial flow impeller(s).

According to one preferred embodiment, the method provided by the present invention comprises introducing neat bromine and a Lewis acid catalyst into a reactor provided with one or more high axial flow impeller(s), which impellers are preferably arranged in the agitator assembly described above, gradually feeding the diphenyl ether starting material into said reactor, during which period additional amounts of bromine may be introduced into the reactor, and allowing the bromination reaction to proceed and reach completion, preferably with heating, under the mixing generated by said axial flow impeller(s). Upon completion of the reaction, the product is recovered by cooling the reaction mixture, destroying the catalyst, distilling excess bromine concurrently with the addition of water, and separating the highly pure solid from the liquid phase, e.g., by filtration.

The parameters of the bromination reaction carried out under the stirring generated by the high axial flow impellers described above may be adjusted by the skilled artisan. It is preferred, however, to carry out the bromination under heating, with the rotation speed of the impeller(s) being in the range of 100 to 120 rpm. The rotation speed of the impeller(s) and the rate of charging the starting material (diphenyl ether) may also be readily adjusted by the skilled artisan; for example, in the large capacity reactor described above, it is possible to convert about 2000 kg of diphenyl ether into highly pure decabromodiphenyl ether using bromine as the solvent within 5 to 10 hours. The completion of the reaction can be monitored by sampling for CG, HPLC and melting point.

Regarding the preparation of decabromodiphenyl ethane, a preferred process provided by the present invention comprises introducing bromine and a Lewis acid catalyst into a reaction vessel, gradually feeding the diphenyl ethane starting material in a molten state into said reaction vessel, wherein the temperature of the reaction mixture during said gradual feed is maintained in the range between 21° C. and reflux temperature, and more specifically, between 50 and 60° C., allowing the bromination reaction to proceed while concurrently providing in-situ a reduced particle size precursor of decabromodiphenyl ethane (by milling the reaction mixture), and upon completion of the reaction, separating from the reaction mixture a crude polybrominated product which contains from 90 to 99% decabromodiphenyl ethane, said product having a particle size distribution characterized by d₅₀ parameter smaller than 11.0 microns, and preferably equal to or less than 10.0 microns. More preferably, the crude product comprises from 95 to 99%, even more preferably 97-99%, decabromodiphenylethane, with a particle size distribution characterized by d₅₀ parameter equal to or less than 10 microns, preferably in the range between 7 and 10 microns. It should be noted that the particle size distribution as reported hereinabove for the crude decabromodiphenylethane refers to the population of the particles separated from the reaction mixture and subjected to washing, before drying and grinding (such grinding is often carried out on the dry particles of the product in order improve its color characteristics). Accordingly, the particle size distribution reported herein for decabromodiphenyethane corresponds to the particles of the crude product as generated by the reaction under the in-situ milling of the invention.

More specifically, upon completion of the reaction, the decabromodiphenyl ethane product is recovered by cooling the reaction mixture, destroying the catalyst, removing excess bromine, and separating the solid from the liquid phase, e.g., by filtration, followed by washing and drying. Subsequently, other work-up steps known in the art (grinding and heat treatment) are applied.

The crude decabromodiphenyl ethane product with the preferred assay and particle size distribution profiles reported hereinabove: namely, a crude product obtainable from a reaction mixture, which product comprises from 95 to 99%, more preferably 97-99% most preferably between 98-99% decabromodiphenylethane, with a particle size distribution characterized by d₅₀ parameter in the range between 7 and 10 microns, forms another aspect of the invention.

### EXAMPLES

### Analytical Methods

(i) Qualitative Gas Chromatography:

Instrument: Hewlett Packard model 5890 with ECD detector

Column: DB-1 (10 m×0.53 mm×1.5 μm)

Heating program: Decabromodiphenylether-150° C., 20° C./min to 300° C., 320° C., 12 minutes; Decabromodiphenylethane-250° C., 20° C./min, final temp. 300° C., 13 min.

Injector: 170° C. for decabromodiphenylether and 270° C. for decabromodiphenylethane

Detector: 350° C.

Sample preparation: Five mg of sample is dissolved in 20 ml of CS₂ One μl is injected.

(ii) Qualitative and Quantitative HPLC:

Instrument: HPLC with UV detector

Column: 5μ ODS (C-18) end-capped Apollo 150×4.6 mm or equivalent

Temperature: Room temperature

Detector: 230 nm

Injector volume: 5 μL

Eluent composition: 90% acetonitrile, 10% water (v/v)

Solvent flow rate: 1.5 ml/min

Sample preparation: 100 mg is dissolved in 25 ml toluene.

Preparation of standards for quantitative HPLC: A stock solution of each component in toluene is suitably diluted. In general, the compositions of the products are expressed as area % (qualitative). For products containing more than 99% decabromodiphenyl ether, the content of the nonabromodiphenyl ether impurity was determined by quantitative (wt %) HPLC.

(iii) Particle Size Distribution was Measured on a Malvern Mastersizer 2000 Instrument.

### Example 1

### Decabromodiphenyl Ether—Simultaneous Bromination and Milling in a Mixed Solvent

To a 1 liter round bottomed flask equipped with a mechanical stirrer, a dropping funnel, a thermocouple and a reflux condenser was added 440 g of solvent mixture (DCM 20%, CBM 40%, and DBM 40% w/w); bromine, 475 g; AlCl₃, 4.3 g; and ceramic beads (1.5-3.5 mm diameter), 814 g.

A solution of diphenyl oxide (42.5 g,) in 20 ml of solvent mixture was dropped into the flask during 70 minutes with stirring while keeping the temperature at 7-13° C. The reaction mixture was refluxed for 4.5 hours, the flask was cooled and 55 ml of water was carefully added to destroy the catalyst. Excess bromine was bleached with sodium bisulfite solution, the aqueous phase was separated and the organic phase was washed with water. The product mixture was passed through a sieve to remove the ceramic beads and the mixture was filtered, washed with water and dried. The product comprised 99.4% decabromodiphenyl ether and 0.1% nonabromodiphenyl ether (in the Examples sometimes abbreviated “Deca” and “Nona”, respectively). Particle size 7.1 microns (d₉₀).

### Example 2 (Comparative)

### Decabromodiphenyl Ether—Bromination without Milling in a Mixed Solvent

The procedure of Experiment 1 was repeated without the ceramic beads present. The product consisted of 94.1% Deca and 5.8% Nona. Particle size 98 microns (d₉₀).

### Example 3

### Decabromodipheayl Other—Increasing the Particle Size of Milled Deca in a Simulated Reaction Mixture after Bromination and Destruction of the Catalyst

The product of Example 1, having a particle size of 7.1 microns (d₉₀) and an assay of 99.4%, was charged to a 1 liter flask followed by 440 g of solvent mixture (DCM 20%, CBM 40%, and DBM 40%); 4.3 g AlCl₃, 50 g of bromine and 50 ml of water to destroy the catalyst. The mixture was refluxed for 5.3 hours, cooled and bleached with bisulfite. The product was filtered, washed with water and dried. The particle size was 28.8 microns (d₉₀), had an assay of 99.7% and was easily filtered.

### Example 4

Decabromodiphenyl Ether—Simultaneous Bromination and Milling in Bromine Solvent

To a 1 liter round bottomed flask equipped with a mechanical stirrer, a dropping funnel, a thermocouple and a reflux condenser was added 1200 g bromine, 6.8 g AlCl₃, and 840 g ceramic beads (1.5-3.5 mm diameter). Molten DPO, 60 g was dropped into the flask from the heated funnel during 1 hour while keeping the temperature at about 6° C. The contents of the flask were heated at reflux for 6.2 hours and were then cooled to room temperature. Water, 100 ml, was carefully added to destroy the catalyst. The excess bromine was then distilled with the simultaneous addition of 165 ml of water. Some residual bromine was bleached with sodium bisulfite solution and the product mixture was passed through a sieve to remove the ceramic beads. The mixture was filtered, washed with water and dried. The product comprised 99.9% Deca and 0.1% Nona. Particle size 14 microns (d₉₀).

### Example 5 (Comparative)

### Decabromodiphenyl Ether—Bromination without Milling in Bromine Solvent

The procedure of Experiment 4 was repeated without the ceramic beads present. The product comprised 98.3% Deca and 1.2% Nona. Particle size 143 microns (d₉₀).

### Example 6

Unmilled Deca with a particle size of about 44 microns (d₉₀), prepared on an industrial scale by a process similar to that described in Example 2, was milled in an Alpine Model C4-60 air jet mill having an air supply of 200 cubic meters per hour at a pressure of about 3 atmospheres and with a Deca flow rate of 3 tons per hour. The milled Deca had a particle size of about 4 microns (d₉₀). Such milled material was used in Example 7.

### Example 7

### Decabromodiphenyl Ether—Bromination of Pre-Milled Deca Precursor

To a 2 liter jacketed reaction vessel equipped with a mechanical stirrer, a dropping funnel, a thermocouple and a reflux condenser was added 500 g of milled Deca (content 97.3%, particle size 3.8 microns (d₉₀)), 1945 g of solvent mixture, 444 g bromine and 22 g AlCl₃. The mixture was heated at reflux for 3.5 hours. Water, 260 ml, was added and the excess bromine was bleached with sodium bisulfite solution. To the thick slurry was added 150 ml of water. The mixture was filtered and the dried solid comprised 99.6% Deca with particle size 38.5 microns (d₉₀).

### Example 8

### Decabromodiphenyl Ether—Bromination of Pre-Milled Deca Precursor on an Industrial Scale

A reactor vessel of 16 cubic meters capacity was charged with 7500 liters of solvent consisting of 12.6% dichloromethane, 32.5% bromochloromethane and 54.9% dibromomethane. Aluminum chloride, 150 kg, 8 tons of pre-milled Deca (Deca content 97.9% with particle size 14 microns (d₉₀)) and 1000 kg of bromine were added. The mixture was heated at reflux for 8 hours and was then bleached with 1340 liters of 38% sodium bisulfite solution. The upper aqueous solution was decanted and the mixture was washed with two 1200 liters portions of water. Sodium hydroxide, 20%, was added to neutralize the mixture which was then centrifuged. The product was dried and was found to contain 99.3% Deca with particle size 42 microns (d₉₀).

### Example 9

### Decabromodiphenyl Ethane—Simultaneous Bromination and Milling in a Mixed Solvent

To a 1 liter round bottomed flask equipped with a mechanical stirrer, a dropping funnel, a thermocouple and a reflux condenser was added 520 g of solvent mixture (DCM 6%, CBM 20%, and DBM 74%), bromine, 539 g; AlCl₃, 9 g; and ceramic beads (1.5-3.5 mm diameter), 840 g.

A 55% solution of diphenyl ethane in DCM (91.1 g,) was dropped into the flask during 30 minutes with stirring while keeping the temperature at 21-26° C. The reaction mixture was refluxed for 6.7 hours, and 120 ml of water was carefully added to destroy the catalyst. Bromine was bleached with sodium bisulfite solution, the aqueous phase was separated and the organic phase was washed with water and was neutralized with 20% NaOH. The product mixture was passed through a sieve to remove the ceramic beads and the mixture was filtered, washed with water and dried. The product comprised 91.4% decabromodiphenyl ethane and 7.8% nonabromodiphenyl ethane. Particle size was 6.1 microns (d₉₀).

### Example 10 (Comparative)

### Decabromodiphenyl Ethane—Bromination without Milling in a Mixed Solvent

The procedure of Example 9 was repeated without the ceramic beads present. The product comprised 80.6% decabromodiphenyl ethane and 18.6% nonabromodiphenyl ethane. Particle size 22 microns (d₉₀).

### Example 11

### The Reduction of Particle Size of a Precursor of Decabromodiphenyl Ether (In Situ Milling) by Stirring with an Impeller

The purpose of the following example is to demonstrate that an impeller may be used for reducing the particle size of an in situ formed precursor of decabrornodiphenyl ether suspended in bromine. The thus obtained reduced particle size precursor of Decabromodiphenyl ether has improved accessibility to the attack of molecular bromine on its aromatic rings, allowing the formation of the desired, highly pure, decabromodiphenyl ether.

A 1 liter flask equipped with a stirrer having an anchor impellor was charged with 200 g of Decabrdmodiphenyl ether (content 97.3%; average particle size 176 microns (d₅₀)) and 125 ml of bromine. The mixture was cooled to 0° C. and was stirred at 500 rpm. After 2 hours the average particle size was 103 microns and after 5 hours 79 microns.

### Example 12 (Comparative)

### Decabromodiphenyl Ether—Bromination of Deca Completely Dissolved in Bromine

The solubility of Deca in bromine was determined as 2.64 g Deca in 100 g bromine at 20° C.

To a 1 liter round bottomed flask equipped with a mechanical stirrer, a dropping funnel, a thermocouple and a reflux condenser was added 22 g of non milled Deca (content 97.1%) and 1642 g bromine to produce a 1.32% solution. AlBr₃, 14.1 g, was added and the mixture was heated at reflux for 5 hours. After cooling to room temperature, water, 250 ml, was carefully added. The bromine was distilled and an additional 520 g of water was added. The solid was filtered and dried and consisted of 99.6% Deca.

### Example 13

### Some Properties of Deca of Different Purities

Some properties and characteristics of interest of the highly pure (99.8%) Deca obtained by the method of the present invention were compared with those of less pure (97.3%) material. The purity levels are expressed in terms of GC area %. The results are given in the table below.

| Property | Deca, 99.8% | Deca, 97.3% |
|---|---|---|
| Melting point (° C.) | 309.6-310 | 307-307.3 |
| DSC, T Fusion 10% (° C.) | 305.7 | 302.4 |

| | |
|---|---|
| XRPD | Both are identical |

The melting point was determined by the capillary method with a Buchi 545 instrument.

X-ray powder diffraction patterns were measured on a Rigaku X-ray Difractometer Ultima Plus instrument with a copper tube at 40 kvolts and 20 mA.

DSC (differential scanning calorimetry) was determined with a Mettler Toledo Star System, at heating rate of 1° C./min.

### Example 14—(Comparative)

### Decabromodiphenyl Ethane—Bromination without Milling in Bromine Solvent

A 2 liter double surface reactor was equipped with a mechanical stirrer, a heated dropping funnel, a thermocouple well and a reflux condenser connected to an HBr trapping system. The reactor was charged with bromine (2.8 kg, 0.9 L, 17.5 mol) and aluminum chloride (13.5 g, 0.1 mol). Molten diphenyl ethane (DPE, 100 g, 0.549 mol) was fed into the reactor during 2 hours with stirring while keeping the temperature at 30-35° C. The reaction mixture was then refluxed for 15 hours.

After the heating period the mixture was cooled to 45° C. and water (50 mL) was carefully added to deactivate the catalyst. More water was added (2 L) and the bromine was distilled off until the temperature reached ˜100° C. The resulting slurry was neutralized with sodium bisulfite and caustic and then withdrawn from the reactor, filtered, washed with water and dried.

The product comprised 89.5% decabromodiphenyl ethane, 4.4% nonabromodiphenyl ethane. Particle size was 27 microns (d₅₀), 53 microns (d₉₀)

### Example 15

### Decabromodiphenyl Ethane—Bromination with Milling in Bromine Solvent

The procedure of Example 14 Was repeated with the addition of 1.6 kg ceramic beads. After bromine separation and neutralization, the resulting slurry was withdrawn from the reactor and the beads remained in the reactor.

The product comprised 98.1% decabromodiphenyl ethane, 1.8% nonabromodiphenyl ethane. Particle size was 10 microns (d₅₀), 27 microns (d₉₀).

1) A process for preparing a polybrominated product of the following structure: 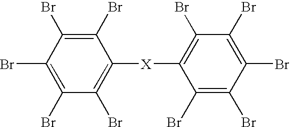
wherein X is —O— or —CH2—CH2—, which comprises brominating a reduced particle size precursor of said polybrominated product in an organic solvent or in bromine as a solvent, wherein the bromination is carried out either concurrently with or subsequent to said particle size reduction, forming the polybrominated product and separating the same from the reaction mixture.

2) A process according to claim 1, wherein the polybrominated product is highly pure decabromodiphenyl ether containing less than 1.0% nonabromodiphenyl ether.

3) A process according to claim 2, wherein the solvent comprises one or more halogenated hydrocarbons.

4) A process according to claim 2, wherein the solvent is bromine.

5) A process according to claim 2, wherein the precursor is a pre-milled, reduced particle size decabromodiphenyl ether material containing not less than 95% decabromodiphenyl ether and more than 1.1% of a nonabromodiphenyl ether impurity.

6) A process according to claim 5, wherein the reduced particle precursor is characterized by d₉₀ value of less than 14μ.

7) A process according to claim 2, which comprises milling the solids in the reaction vessel concurrently with the bromination reaction.

8) A process according to claim 7, which process comprises brominating diphenyl oxide in a solvent or in bromine while subjecting the reaction mixture to milling, thereby providing in-situ a reduced particle size precursor of decabromodiphenyl ether.

9) A process according to claim 8, wherein the bromination reaction is carried out in bromine as a solvent and the milling is accomplished by stirring the reaction mixture using one or more high axial flow impellers(s).

10) A process according to claim 9, which comprises introducing bromine and a Lewis acid catalyst into a reactor provided with one or more high axial flow impeller(s), gradually feeding the diphenyl ether starting material into said reactor, allowing the bromination reaction to proceed and reach completion under heating and under the mixing generated by said axial flow impeller(s), cooling the reaction mixture, destroying the catalyst, distilling excess bromine concurrently with the addition of water and separating the highly pure solid from the liquid phase.

11) A process according to claim 2, which comprises passing the reaction mixture through a milling device placed outside the reaction vessel, whereby a reduced particle size precursor of decabromodiphenyl ether is formed, delivering the thus formed reduced particle size precursor to the reaction vessel and completing the bromination reaction to produce the highly pure decabromodiphenyl ether.

12) A process according to claim 2, which comprises isolating solids from the liquid phase prior to the completion of the bromination reaction, milling said solids, delivering the same to the reaction vessel and completing the bromination reaction to produce the highly pure decabromodiphenyl ether.

13) A process according to claim 2, which further comprises heating the reaction mixture following the formation of the polybrominated product, thereby enlarging the size of the particles of said polybrominated product, and separating the same from the liquid phase.

14) A process according to claim 1, wherein the polybrominated product is decabromodiphenyl ethane containing less than 10% underbrominated derivatives.

15) A process according to claim 14, wherein the solvent comprises one or more halogenated hydrocarbons.

16) A process according to claim 14, wherein the solvent is bromine.

17) A process according to claim 14, which comprises brominating diphenyl ethane in one or more halogenated hydrocarbons as solvent or in bromine as a solvent while subjecting the reaction mixture to milling, thereby providing in-situ a reduced particle size precursor of said decabromodiphenyl ethane.

18) A process according to claim 17, wherein bromine is used as the solvent.

19) A process according to claim 17, wherein the polybrominated product that is separated from the reaction mixture comprises between 90% and 99% decabromodiphenyl ethane compound.

20) A process according to claim 18, wherein the polybrominated product that is separated from the reaction mixture comprises between 95% and 99% decabromodiphenyl ethane compound.

21) A process according to claim 18, which comprises introducing bromine and a Lewis acid catalyst into a reaction vessel, gradually feeding the diphenyl ethane starting material in a molten state into said reaction vessel, wherein the temperature of the reaction mixture during said gradual feed is maintained in the range between 21° C. and reflux temperature, allowing the bromination reaction to proceed while concurrently providing in-situ a reduced particle size precursor of decabromodiphenyl ethane, and upon completion of the reaction, separating from the reaction mixture a crude polybrominated product which contains from 90 to 99% decabromodiphenyl ethane, said product having a particle size distribution characterized by d₅₀ parameter equal to or less than 10 microns.

22) A process according to claim 21, wherein the product contains from 95 to 99% decabromodiphenyl ethane, said product having a particle size distribution characterized by d₅₀ parameter in the range between 7 and 10 microns.

23) A process according to claim 14, which comprises passing the reaction mixture through a milling device placed outside the reaction vessel, whereby a reduced particle size precursor of decabromodiphenyl ethane is formed, delivering the thus formed reduced particle size precursor to the reaction vessel and completing the bromination reaction to produce the decabromodiphenyl ethane containing less than 10% underbrominated derivatives.

24) A process according to claim 14, which comprises isolating solids from the liquid phase prior to the completion of the bromination reaction, milling said solids, delivering the same to the reaction vessel and completing the bromination reaction to produce decabromodiphenyl ethane containing less than 10% underbrominated derivatives.

25) A crude polybrominated reaction product which comprises from 95 to 99% decabromodiphenylethane, with a particle size distribution characterized by d₅₀ parameter in the range between 7 and 10 microns.

26) A crude polybrominated reaction product of claim 25, comprising from 97 to 99% decabromodiphenylethane.
